(19) United States
(12) Patent Application Publication
(10) Pub. No.: US 2023/0100362 A1
(43) Pub. Date: Mar. 30, 2023

(54) Conjugate Vaccines Containing Hydrazine-PEG-Hydrazine Linkers

(71) Applicant: Subhash V. Kapre, Redmond, WA (US)

(72) Inventor: Subhash V. Kapre, Redmond, WA (US)

(21) Appl. No.: 18/074,660

(22) Filed: Dec. 5, 2022

Related U.S. Application Data
(63) Continuation-in-part of application No. 17/733,473, filed on Apr. 29, 2022, now abandoned.
(60) Provisional application No. 63/181,476, filed on Apr. 29, 2021.
(60) Provisional application No. 63/270,210, filed on Oct. 21, 2021.

(51) Int. Cl. A61K 39/215; A61K 39/39; A61P 31/14

(52) U.S. Cl. CPC A61K 39/215; A61K 39/39; A61P 31/14; A61K 2039/627

(57) ABSTRACT

This invention is directed to compositions, vaccines, and methods for the manufacture and administration of immunogenic compositions containing one or more antigenic regions of a pathogen coupled via hydrazine-PEG-hydrazine linkers to a carrier, and to immunogenic compositions that are vaccines. Immunogenic compositions of the invention may contain multiple epitopes obtained or derived from multiple variants of the same or different infectious pathogens which may be bacterial and/or viral. The invention is also directed to methods for the manufacture or immunogenic composition, and methods for the prevention and treatment of pathogenic infections by administration of immunogenic compositions disclosed herein.

### REFERENCE TO RELATED APPLICATIONS

This application is a continuation-in-part of U.S. application Ser. No. 17/733,473 filed Apr. 29, 2022, now abandoned, which claims priority to U.S. Provisional Application No. 63/181,476, filed Apr. 29, 2021, and U.S. Provisional Application No. 63/270,210, filed Oct. 21, 2021, the entirety of each of which is specifically incorporated by reference.

### BACKGROUND

### 1. Field of the Invention

This invention is directed to methods and devices for the manufacture and use of conjugate immunogenic compositions and vaccines containing hydrazine-PEG-Hydrazine linkers, and to the conjugate immunogenic compositions and vaccines.

### 2. Description of the Background

A capsular polysaccharide (CPS) is a key virulence determinant and generally insufficiently immunogenic to induce a T cell-dependent immune response in infants and children. Conjugation of a carrier protein to CPS can induce an immune response that undergoes class switching. Accordingly, a 7-valent (PCV-7, Pfizer Inc., USA), a 10-valent (Synflorox-10, GSK Vaccines) and a 13-valent pneumococcal conjugate vaccine (PCV-13, Pfizer Inc., USA) have been developed to efficiently prevent the incidence of IPDs. Reductive amination chemistry and cyanylation chemistry has been widely used to prepare the conjugate vaccines.

The SARS-CoV-2 strain has undergone several mutations after being in circulation since 2019. In the initial stages the binding with the ACE receptor to get an entry into human cells seems was not effective and so even with a large number of virus particles entering the respiratory system it was able to affect mainly older age population who had a diminished immune response and was not showing up in younger age groups.

After the advent of vaccine doses, the older age group were able to protect first, and this removed the group as a major group showing infections. In the years since identification of the virus, multiplication has shown major mutations causing a new behavior. Effectivity of binding to the ACE receptor has enhanced, and the mutation caused the earlier antibodies are less effective due to a change in the epitopes. This has resulted in high infectivity rates with lessor number of viral particles. The major population that has now fallen a prey is younger age groups. This has 11N g caused number of infections that earlier wave and become a major global problem. Thus, the need for an effective treatment is present and increasing, especially as hospitalizations increase.

Coronavirus severe acute respiratory syndrome 2 (SARS-CoV-2) has infected more than 219 million people with 4.55 million deaths worldwide. The World Health Organization (WHO) declared the SARS-CoV-2 outbreak as a global pandemic in March 2020. Vaccines and other treatments are desperately needed to combat the spread. One area research that has received little attention is the development of conjugate vaccines against SARS-CoV-2. Covalently attaching an antigen onto a carrier protein can provide a significantly higher and longer lasting immune response compared to the antigen alone.

The non-toxic mutant of diphtheria toxin, cross-reactive material 197 (CRM197), has a long and successful history as the carrier protein of conjugate vaccines. CRM197-based vaccines have been FDA approved to combat pneumococcal, H. influenzae b, as well as meningitis ACWY infections. The power of the CRM197 carrier protein for antigens comes from CRM197's ability to directly target antigen presenting cells (APCs). The CRM197 binds to the heparin-binding EGF-like growth factor (HB-EGF) on the surface of the APC to initiate endocytosis. The endocytosis of the antigen by the APC is not left to chance since the CRM197 directly transports the antigen directly into the APC. Another advantage of using CRM197 is that the T cell dependent mechanism is active within infants shortly after birth. The spike glycoprotein (S-protein) of the coronavirus, has undergone intense research as a vaccine candidate for both SARS-CoV-1 and SARS-CoV-2. The SARS-CoV-2 strain has undergone several mutations after being in circulation since 2019.

In the initial stages, binding with the ACE receptor, which provides entry into human cells, was not very effective. Even with many virus particles entering the respiratory system, infection was mainly achieved in older age populations who had a diminished immune response, which was not evident in younger age groups. After the advent of vaccines, the older age group were able to protect first, and this removed the group as a major group showing infections. In the years since identification of the virus, multiplication has shown major mutations causing a new behavior. Effectivity of binding to the ACE receptor has enhanced, and the mutation caused the earlier antibodies are less effective due to a change in the epitopes. This has resulted in high infectivity rates with lessor number of viral particles. The major population that has now fallen a prey is younger age groups. This has caused number of infections that earlier wave and become a major global problem. Thus, the need for an effective treatment of infections caused by coronavirus is present and increasing, especially as hospitalizations increase.

### SUMMARY OF THE INVENTION

The present invention overcomes the problems and disadvantages associated with current strategies and designs and provides new compositions and methods

One embodiment of the invention is directed to an immunogenic composition comprised of one or more antigenic regions of a pathogen coupled to one or more carrier molecules via a hydrazine-PEG-hydrazine linker. Preferably the one or more antigenic regions comprise one or more epitopes of a peptide, a polysaccharide, a protein, a lipid, a fatty acid, or a combination thereof. Preferably the pathogen comprises a virus, a bacterium, a fungus, and/or a parasite, such as, for example, coronavirus and SARS-COV2. Preferably the carrier molecule is a protein such as, for example, tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof. Preferably immunogenic composition comprise two or more regions of different variants of SARS-CoV-2. Also preferably, the immunogenic composition is a vaccine that treats or prevents an infection. Preferably the composition comprises an adjuvant such as, for example, an aluminum compound, or the adjuvant does not contain aluminum. A preferred adjuvant comprises CpG.

Another embodiment of the invention is directed to a method of manufacture of an immunogenic composition, comprising providing one or more antigenic regions of SARS-CoV-2; and coupling the antigenic regions of SARS-CoV-2 to a carrier molecule via a hydrazine-PEG-hydrazine linker.

Another embodiment of the invention comprises methods for preventing or treating a SARS-CoV-2 infection comprising administering to a subject an immunogenic composition comprised of one or more antigenic regions of a pathogen coupled to one or more carrier molecules via a hydrazine-PEG-hydrazine linker. Upon administration, composition create uniform high immune response and a decrease in the antibody response to the carrier protein. This is done by using PEGylation and by having the same PEG molecule connect with the polysaccharides as well as the carrier protein to avoid PEG overload.

Other embodiments and advantages of the invention are set forth in part in the description, which follows, and in part, may be obvious from this description, or may be learned from the practice of the invention.

### DESCRIPTION OF THE FIGURES

FIG. 1 Beta binding IgG titers—monovalent vs. bivalent.

FIG. 2 Delta binding IgG titers—monovalent vs. bivalent.

FIG. 3 nAB titers to beta and omicron—2 weeks post boost.

FIG. 4 nAB titers to beta and omicron—4 weeks post boost.

FIG. 5 Pseudovirus neutralization titers at day 49.

FIG. 6 SEC-HPLC profile of Beta variant Spike S-protein conjugated with Pegylated CRM197 using carbodiimide chemistry (EDC/sNHS).

FIG. 7 Animal Immunogenicity study in BALB/c mice.

FIG. 8 Various formulations of SPIKE-S-PROTEIN-CRM197 conjugate with Alum and Th1-Specific Adjuvant.

FIG. 9A Elisa comparisons of 1 dose regiments of wt S-2P with IgG values.

FIG. 9B Elisa comparisons of 2 dose regiments of wt S-2P with IgG values.

FIG. 9C Elisa comparisons of 1 dose regiments of beta S-2P with IgG values.

FIG. 9D Elisa comparisons of 2 dose regiments of beta S-2P with IgG values.

FIG. 10 Titers of bound antibodies as detected by Elisa.

### DESCRIPTION OF THE INVENTION

SARS-CoV-2 strain has been in circulation around the world since at least 2019. In the initial stages of infection, the virus binds with the ACE receptor which provides entry into human cells. With the advent of new vaccines, older age group were protected first, and this removed the older age group as a major group showing infections. In the years since identification of the virus, multiplication has shown major mutations causing a new behavior. Mutations showed that binding to the ACE receptor was enhanced and antibodies were found to be less effective due to a change in viral epitopes. This has resulted in high infectivity rates with smaller numbers of viral particles. The major population that has now fallen a prey is younger age groups and is a major global problem.

It has been surprisingly discovered that vaccines created through conjugation can economize on costs, and also create effective vaccine solutions by making a multicomponent vaccine to address infection caused by different variants. Coupling methods using a PEG-linker increase the vaccine's efficacy and not only enhance the antibody numbers, but also create a longer lasting effect. An added benefit is that such vaccines can be administered to children and infants, which can require lower dose volumes of vaccine (e.g., 1 ml or less, ½ ml or less).

The present disclosure is directed to PEGylated compounds, of immunogenic compositions, and vaccines comprising carrier protein compounded to antigenic regions of a pathogen. Pathogens that may be prevented or treated include viral and/or bacterial infections, and parasitic and/or fungal diseases. Pathogens include, for example, Streptococcus spp., Pseudomonas spp., Shigella spp., Yersinia spp. (e.g., Y. pestis), Clostridium spp. (e.g., C. botulinum, C. difficile), Listeria spp., Staphylococcus spp., Salmonella spp., Vibrio spp., Chlamydia spp., Gonorrhea spp., Syphilis spp., MRSA, Streptococcus spp., Escherichia spp. (e.g., E. coli), Pseudomonas spp., Aeromonas spp., Citrobacter spp (e.g., C. freundii, C. braaki), Proteus spp., Serratia spp., Klebsiella spp., Enterobacter spp., Chlamydophila spp., Mycobacterium spp., MRSA (Methicillin-resistant Staphylococcus aureus), Mycoplasma spp. (e.g., MTB), and Ureaplasma spp (e.g., U. parvum, U. urealyticum). Virus on which the immunogenic composition can be formed include, for example, influenza virus, corona virus and variants (e.g., Covid-19, MERS-COV. SARS-COV2, alpha, beta, delta, gamma, omicron, epsilon), respiratory syncytial virus, parainfluenza virus, adenovirus, rhinovirus, human metapneumovirus, and enterovirus, Rubella virus, Hepatitis virus, Herpes Simplex virus, retrovirus, varicella zoster virus, human papilloma virus, parvovirus, HIV, and zika virus. Parasitic infections on which the method of the invention can be applied include, for example, Plasmodium spp., Leishmania spp., Guardia spp., endoparasites, protozoan, and helminth spp. Fungal infections to which the methods of the invention can be applied include, for example, Cryptococci, aspergillus and candida. Diseases that can be prevented or treated include, for example, sepsis, colds, flu, gastrointestinal infections, sexually transmitted diseases, immunodeficiency syndrome, nosocomial infections, Celiac disease, inflammatory bowel disease, inflammation, multiple sclerosis, auto-immune disorders, chronic fatigue syndrome, Rheumatoid arthritis, myasthenia gravis, Systemic lupus erythematosus, and infectious psoriasis.

The antigenic region of the pathogen may comprise a peptide, a protein, a polysaccharide such as a capsular polysaccharide, a lipid, a fatty acid, or a combination thereof. Preferably the antigenic region comprises one or more epitopes of coronavirus proteins spike (S), envelope (E), membrane (M), and nucleocapsid (N), and/or influenza virus proteins HA, NA, and M2e. Preferred epitopes comprise conserved regions of bacteria or viral pathogens. An epitope of the antigenic region may be of any sequence and size, but is preferable composed of natural amino acids more than 5 amino acids in length but less than 100 amino acids in length, preferably less than 80, preferably less than 70, preferably less than 60, preferably less than 50, preferably less than 40, preferably less than 30, preferably between 5 and 25 amino acids in length, preferably between 8 and 20 amino acids in length, and more preferably between 5 and 15 amino acids in length. Linkers are preferably PEGylate coupling the antigenic region with a carrier molecule. Linkers preferably comprise PEG and two hydrazine functional groups cable of covalently compounding with both carrier molecule as well as the antigenic region such as a peptide or polysaccharide. This creates a new class of covalently compounded PEG products that have the additional effect of PEG on their properties compared to conjugates made by established methods. PEG has an additional enhancing effect on the immunogenicity of antigenic region compared to regular conjugates and a depressing effect on the immune response of carrier molecule. As the compounds of the invention contain polysaccharide coupled to PEG which is coupled to carrier, there is no conjugation between polysaccharide and protein. Preferably the linker comprises a hetero- or homo-bifunctional or multifunctional linker such as, for example, NH₂—PEG-NH₂/NHS, NHS/NH₂—PEG-COOH, Mal-PEG-NH₂, Mal-PEG-NHS, CHO-PEG-CHO, SH-PEG-NH₂, ADH, HZ-PEG-HZ, SMPH, SMCC, 4-Arm-PEG-NH₂. The disclosures herein provide a universal method of covalent PEGylated compounds with an increased immune response, which is unaltered in spite of increase in serotypes, and broader utility (e.g., to additional serotypes). This unexpected beneficial observation is critically important in developing immunogenic compounds such as vaccines.

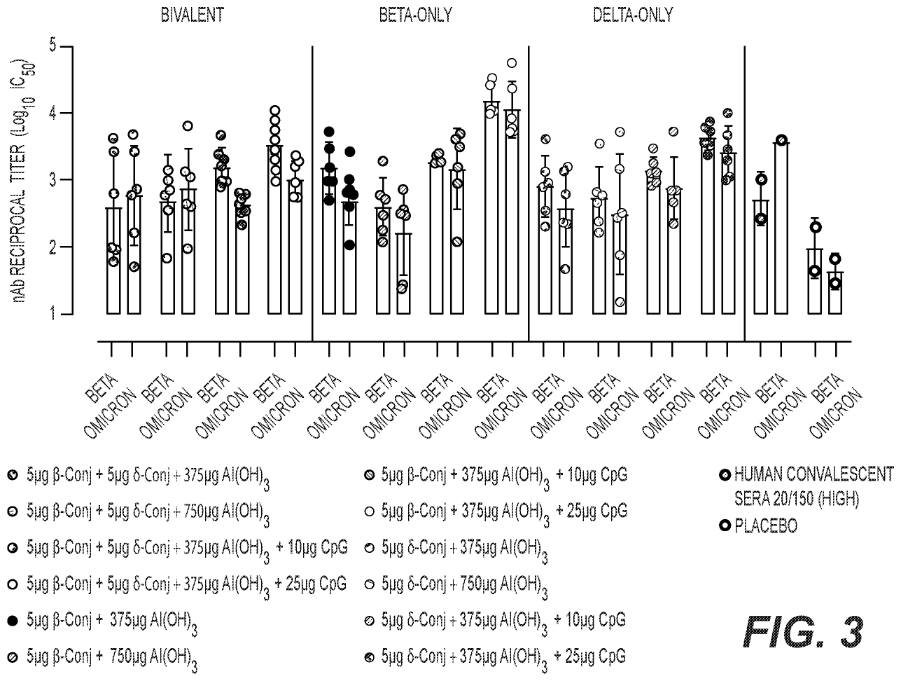

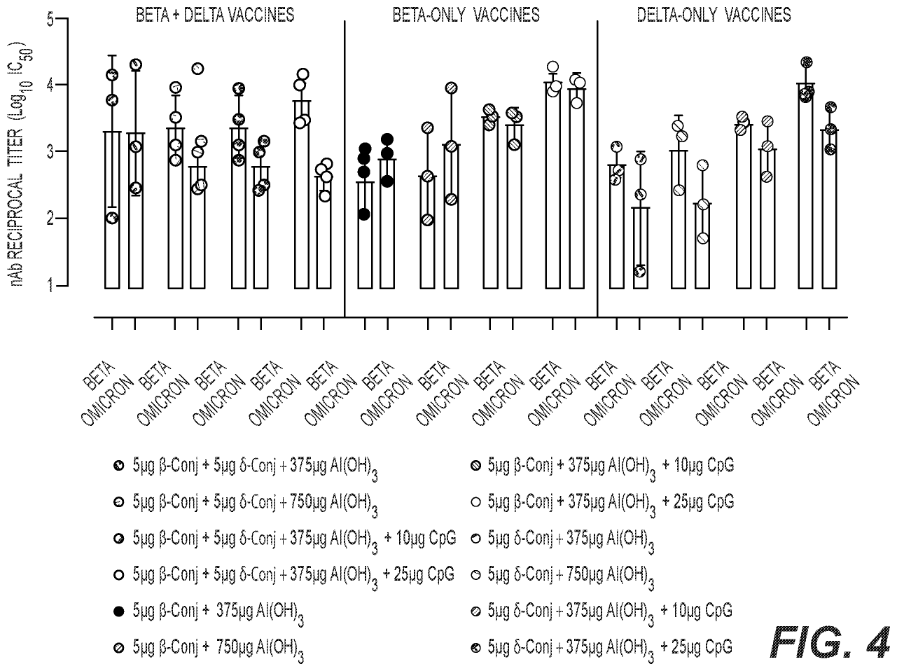

An unexpected and surprising advantage of the immunogenic compositions disclosed herein is their effectiveness is treating multiple variants of the same pathogen. By way of example, an immunogenic composition of the disclosure comprising a bivalent structure of SAR-COV2 shows similar titers against the beta variant and the omicron variant (see FIGS. 3 and 4).

Protection against the pathogenic disease is obtained by antibodies produced against key epitopes of the composition. By PEGylation, the response observed is increased as compared to non-PEGylated compounds. This means that the high antibodies observed after administration of the PEGylated form of the vaccine will fall slowly. This result can eliminate a need for multiple numbers of injections, thus saving cost as well as pain to infants and others caused by multiple injections, and in addition, makes protection more widely available, especially for those unable to return for repeated injections.

As disclosed herein, four parameters have been introduced to minimize the disadvantages of conventional vaccines:
Component (e.g., polysaccharide) size is preferably 10-50KDa.
Cross-reactive polysaccharides concurrent covalent connection to carrier protein.
Two or more cross reactive serotypes are covalently compounded concurrently with carrier proteins.
A long hetero- or homo-bifunctional PEG spacer arm is preferably from 2-40 Å (but may be from 2-40 Å, 4-40 Å, 10-40 Å, 20-40 Å, 9-20 Å, 5-20 Å, 5-30 Å).

These four parameters taken together are profoundly effective to increase the polysaccharide/protein ratio in the covalent compound to reduce carrier protein load, and to provide several folds of increase in immunogenicity and avidity.

It has been surprisingly discovered that immunogenic compositions such as vaccines created through conjugation can economize on costs, and also create effective vaccine solutions by making a multicomponent vaccine to address different variants. Conjugation methods that include a PEG-linker enhance the antibody numbers and create a longer lasting effect. An added benefit is that such vaccines can be administered to children and infants. Covalently attaching a protein antigen onto a carrier protein can provide a significantly higher and longer lasting immune response compared to the protein antigen alone.

This invention relates to materials and method of conjugate immunogenic compositions and vaccines. In particular, compositions may contain pegylated carrier protein and spike protein of SARS-CoV-2. This disclosure includes methods for the manufacture and use of the conjugate immunogenic compositions and vaccines containing pegylated linkers disclosed. Immunogenic compositions of the invention may contain multiple epitopes obtained or derived from multiple variants of the same or different infectious pathogens which may be bacterial and/or viral.

Preferably, the compositions contain high molecular weight constructs with recombinant RBD conjugated to tetanus toxoid induce a potent immune response in laboratory animals. Advantages of the immunization with the viral antigen coupled to pegylated carrier protein include having a predominant IgG immune response and long-term specific B-memory cells. This disclose demonstrates that subunit protein based conjugate vaccines can be an alternative for COVID-19, paving the way for other viral conjugate vaccines based on the use of viral proteins involved in the infection process.

Protection against infections disease is obtained by antibodies produced against the key epitopes. By PEGylation, the response observed is increased as compared to non-PEGylated compounds. This means that the high antibodies observed after administration of the PEGylated form of the vaccine will fall slowly. This is an entirely unexpected and extremely beneficial outcome. This result eliminates any need of multiple injections saving cost as well as pain to infants and others caused by multiple injections, and in addition, makes protection more widely available, especially for those unable to return for repeated injections.

Disclosed herein are promising vaccine candidates based on this high molecular weight conjugate with several copies of Spike-S-Proteins per molecular unit. Chemically conjugated constructs and the immunogenic effect of conjugating viral proteins such as Spike-S-Protein to a Pegylated protein carrier have been assessed for SARS-CoV-2. The Spike-S-protein-[PEG bridge]-CRM197 conjugate induces a potent immune response in laboratory animals.

The following examples illustrate embodiments of the invention, but should not be viewed as limiting the scope of the invention.

### EXAMPLES

### Example 1 Formulation of Composition Containing S2P and Hydrazine PEG

An immunogenic composition was formulated containing hydrazine PEG hydrazine with two protein entities, the Covid Spike Protein (S2P) and CRM 197. Different spike proteins representing different mutants were used in experiments which started with Wuhan S2P. The mutation in the Wuhan strain resulted in Beta, Delta, and Omicron as the infecting mutant strains.

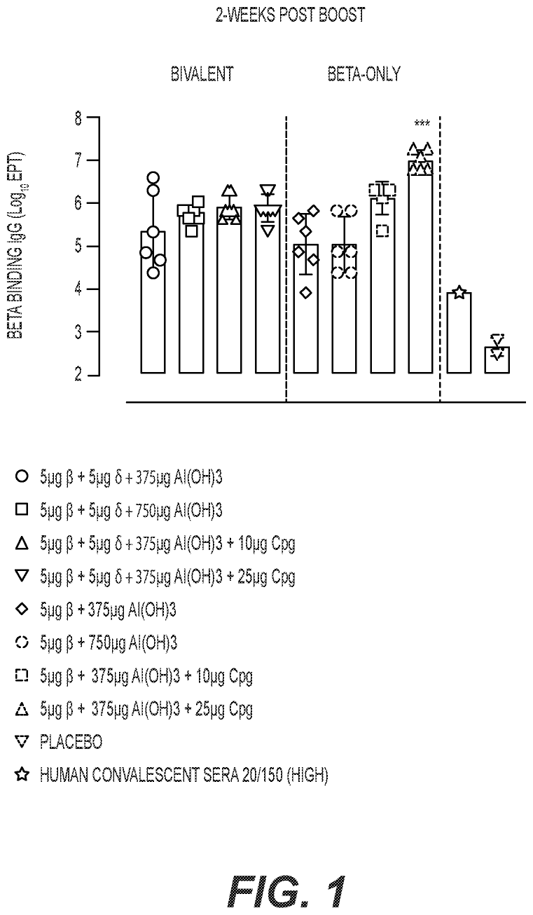

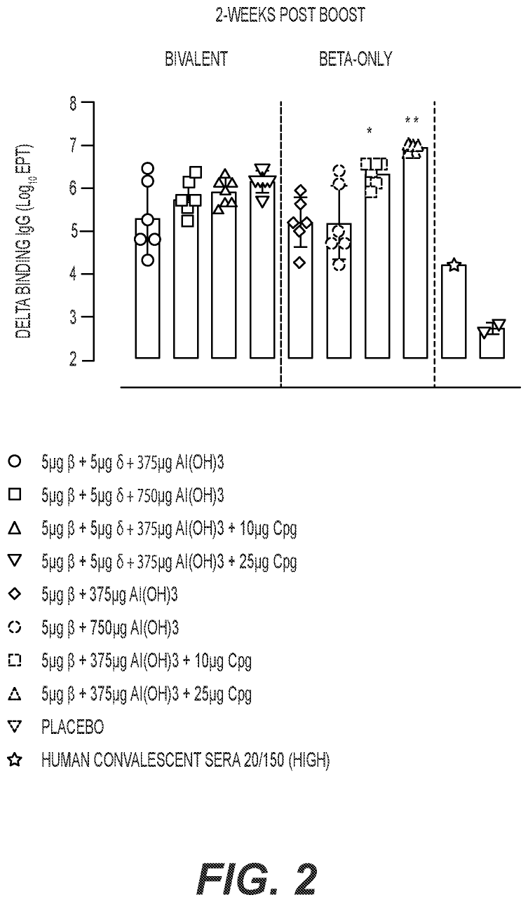

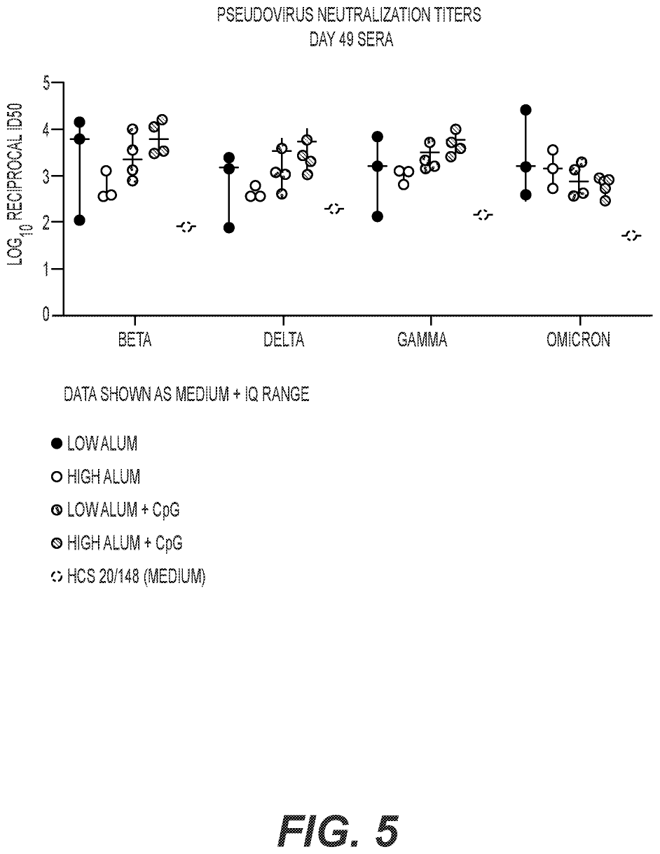

To understand the conjugate-based antibodies, experiments were first done with Wuhan S2P and later with Beta and Delta. The results of virus neutralizing antibodies indicated that Beta S2P conjugate demonstrated a wide range of antibodies capable of showing neutralizing power to all variants. The conjugate also showed a high level of antibodies indicating it is a powerful vaccine for protection against SARS Covid-2. The tests were done with different concentrations of alum and CpG (cytosine phosphoguanine which is a synthetic form of DNA that mimics bacterial and viral genetic material) or CpG oligodeoxynucleotides (ODNs) as adjuvants and the results show that the technology of making a conjugate with the hydrazine-PEG-hydrazine linker makes a vaccine powerful against the various mutants. As depicted in FIGS. 1-4, the bivalent form of the vaccine showed comparably beta binding IgG as compared to the monovalent form (beta only) and irrespective of the adjuvant. FIG. 5 graphically depicts broad neutralization of beta, delta, gamma, and omicron variants of SARS-CoV-2 strains. The adjuvant activity of CpG involves activation of components of both the innate and adaptive immune systems. B cells activated by CpG increase cellular proliferation including CD40, CD80, and MHC II expression as well as their ability to stimulate T cells.

### Example 2 Spike-S-Protein Expression in CHO Cells and Analytical Characterization

CHO cells are transformed with a viral vector containing the Spike-S protein and maintained at 37° C. for a period of time in 20% (v/v) serum. Cells are collected, washed and the Spike-S-protein isolated. Purity is determined on ELISA.

### Example 3 Conjugation of Spike-S-Protein of SARS-COV-2 to Carrier Protein CRM197

Conjugation process design involves conjugating several copies of the SARS-COV-2 to a carrier protein. A high molecular weight construct is obtained displaying multivalent SARS-COV-2. This protein complex is well exposed and available for immune recognition.

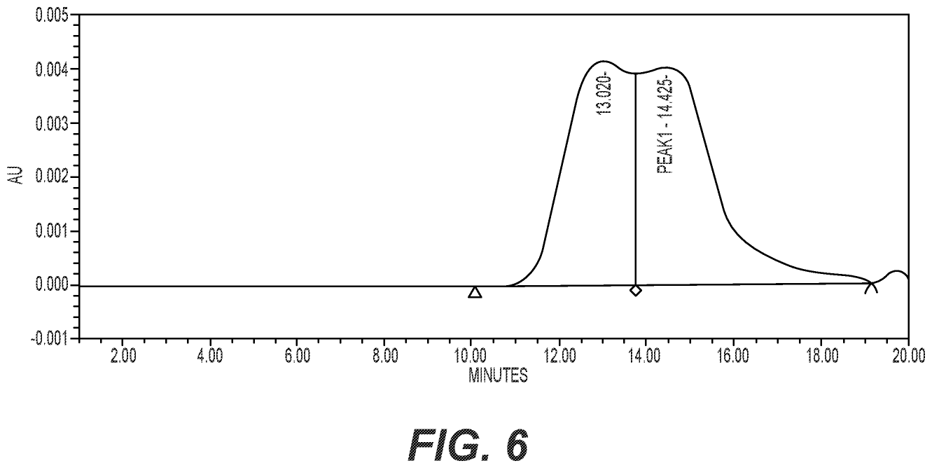

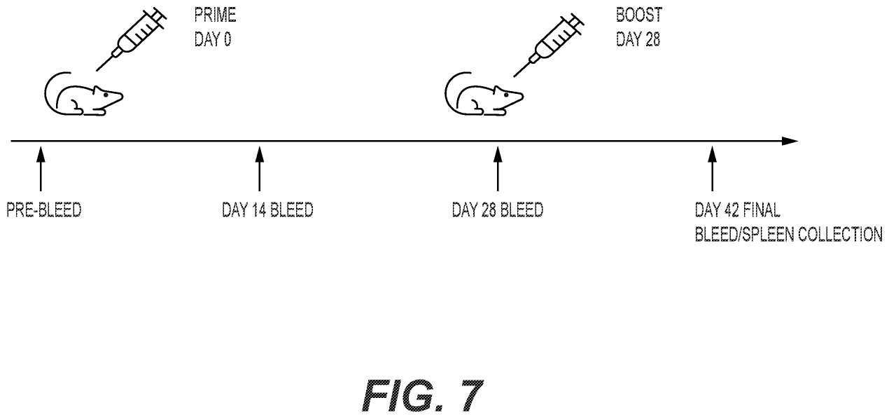

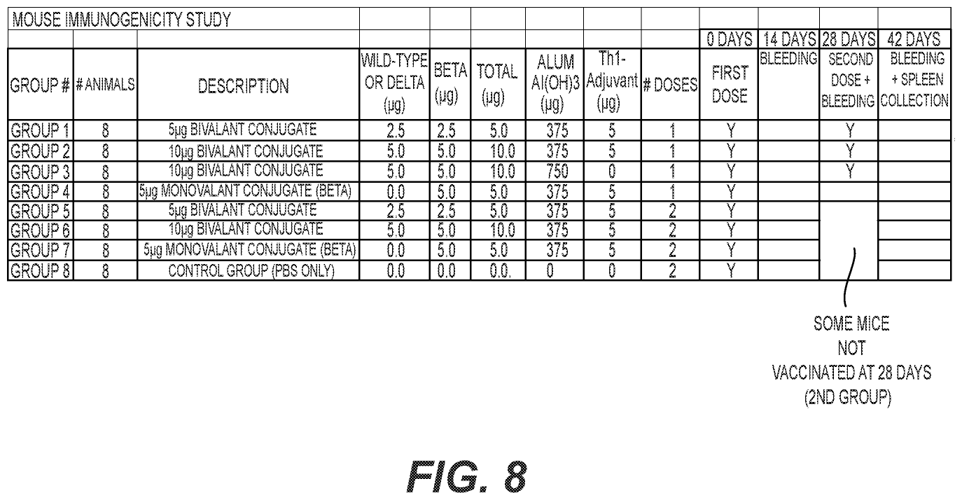

CRM197 was activated with an average of 4-6 pegylated hydrazide groups per mol of CRM197 by reaction with EDC/sNHS followed by reaction with 5 equivalents of EDC/sNHS activated Spike-S-proteins to produce conjugates bearing 2 or 4 mol respectively of Spike-S-proteins per mol of CRM197. The (Spike-S-proteins)2-4-CRM197 conjugate was produced in 60%-70% yield, respectively, and characterized by SE-HPLC (see FIG. 6). Immunization of BALB/c mice (see FIG. 7) with the several different immunogens (see FIG. 8) induced a strong IgG Spike-S-protein-specific immune response detected by enzyme-linked immunosorbent assay (ELISA).

SPIKE-S-PROTEIN-CRM197/alum was compared with SPIKE-S-PROTEIN alone and with SPIKE-S-PROTEIN-CRM197/alum. After the first dose (T14) SPIKE-S-PROTEIN-CRM197/alum induced the highest level of anti-SPIKE-S-PROTEIN antibodies. After the second dose (T28) immunogen adsorbed in alum elicited stronger anti-SPIKE-S-PROTEIN IgG levels than SPIKE-S-Protein/alum (T42).

The high and homogeneous early response for SPIKE-S-PROTEIN-CRM197/alum is an important attribute for a vaccine in pandemic times. At day 14, before the second dose, the response was very high even for the lowest dosage A biased Th2 immune response observed for SPIKE-S-PROTEIN-CRM197/alum and SPIKE-S-PROTEIN/alum while SPIKE-S-PROTEIN-CRM197/alum+Th1-specific Adjuvants displayed more balanced Th1/Th2 immunity.

### Example 4 Animal Studies Results (Total IgG and Neutralization Antibody)

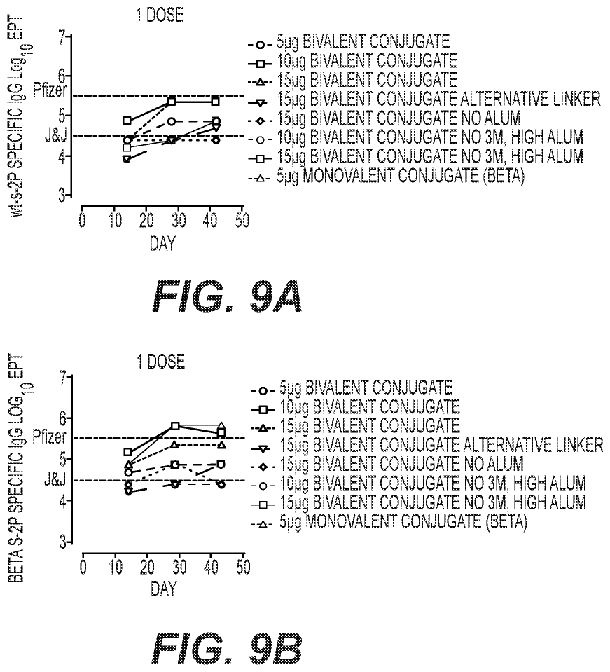

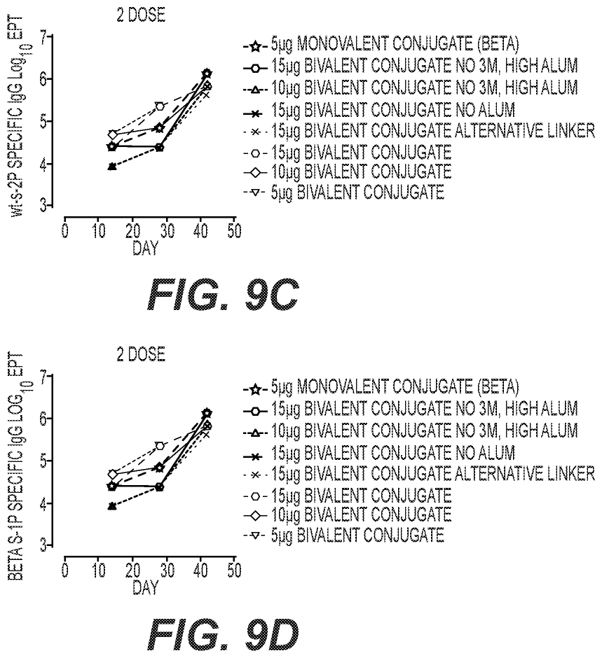

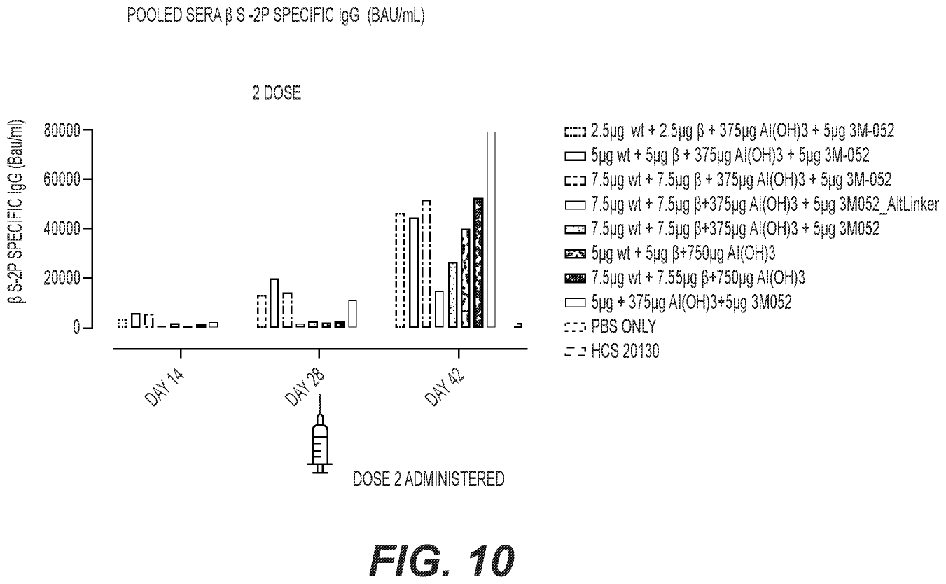

Blood was collected from the orbits of mice on days 0, 14, 28 and 42 of immunization, and the serum was separated and evaluated for binding antibody titer and neutralizing antibody potency, respectively. The ELISA results of mouse sera on day 42 showed (see FIGS. 9 and 10) that antibody titers were induced in these formulations plus adjuvant groups. The serum of mice was isolated after blood sampling on day 42 and the titers of bound antibodies were detected by ELISA (see FIG. 10). The results showed that bound antibodies were detected in all experimental groups, and similar to the serum results on day 14, higher antibody levels were induced in these formulation plus adjuvant groups. In addition, the ELISA simultaneously detected the levels of binding antibodies in mouse sera against the wild-type virulent strain (RBD-WT) and different variants of Beta (SA), Delta Spike-S-proteins. These results show that the sera induced higher levels of binding antibodies against the wild-type and different variants of Spike-S-proteins all had high levels of bound antibodies (see FIG. 9).

Serum neutralizing antibody potency was measured by SARS-CoV-2 live virus neutralization assay using mouse sera isolated on day 42. The results showed that all formulation groups had the neutralizing potency and highest in this formulation plus Adjuvants groups.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. An immunogenic composition comprised of one or more antigenic regions of a pathogen coupled to one or more carrier molecules via a hydrazine-PEG-hydrazine linker.

2. The immunogenic composition of claim 1, wherein the one or more antigenic regions comprise one or more epitopes of a peptide, a polysaccharide, a protein, a lipid, a fatty acid, or a combination thereof.

3. The immunogenic composition of claim 1, wherein the pathogen comprises a virus, a bacterium, a fungus, and/or a parasite.

4. The immunogenic composition of claim 3, wherein the virus comprises coronavirus.

5. The immunogenic composition of claim 4, wherein the coronavirus comprises SARS-COV2.

6. The immunogenic composition of claim 1, wherein the carrier molecule is a protein.

7. The immunogenic composition of claim 6, wherein the protein comprises tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof.

8. The immunogenic composition of claim 1, wherein the one or more antigenic regions comprise regions of multiple variants of SARS-CoV-2.

9. The immunogenic composition of claim 1, further comprising an adjuvant.

10. The immunogenic composition of claim 9, wherein the adjuvant comprises aluminum.

11. The immunogenic composition of claim 9, wherein the adjuvant does not contain aluminum.

12. The immunogenic composition of claim 9, wherein the adjuvant comprises CpG.

13. The immunogenic composition of claim 1, which is a vaccine.

14. A method of manufacture of an immunogenic composition, comprising:
providing one or more antigenic regions of SARS-CoV-2; and
coupling the antigenic regions of SARS-CoV-2 to a carrier molecule via a hydrazine-PEG-hydrazine linker.

15. A method for preventing or treating a SARS-CoV-2 infection comprising:
administering to a subject an immunogenic composition comprised of one or more antigenic regions of a pathogen coupled to one or more carrier molecules via a hydrazine-PEG-hydrazine linker.

16. An immunogenic composition comprising:
one or more peptides, proteins and/or glycoproteins;
one or more carrier proteins conjugated to the one or more peptides, proteins, and/or glycoproteins; and
one or more pegylated linker between the one or more carrier proteins and/or the one or more peptides, proteins and/or glycoproteins.

17. The immunogenic composition of claim 16, which comprises two or more variants of a pathogenic organism.

18. The immunogenic composition of claim 16, further comprising an alum adjuvant.

19. The immunogenic composition of claim 18, wherein the alum adjuvant comprises an alum salt.

20. The immunogenic composition of claim 19, wherein the alum salt boosts a Th1 response in a host.

21. The immunogenic composition of claim 16, further comprising a non-alum adjuvant.

22. The immunogenic composition of claim 16, wherein the one or more spike peptides, proteins, and/or glycoproteins comprise peptides, proteins, and/or glycoproteins of one or more strains of a SARS-CoV-2 virus.

23. The immunogenic composition of claim 16, which is a vaccine.

24. A method of manufacture of the immunogenic composition of claim 16, comprising:
providing epitopes of two or more variants of a pathogenic organism; and
conjugating the epitopes to the one or more peptides, proteins and/or glycoproteins.

25. A method for treating or preventing an infection comprising administering the immunogenic composition of claim 16 to a patient.
